(19) United States
(12) Patent Application Publication
(10) Pub. No.: US 2018/0193059 A1
(43) Pub. Date: Jul. 12, 2018

(54) AUTOMATIC HAIR FOLLICLE TRANSPLANT APPARATUS

(71) Applicants: ELECTRONICS AND TELECOMMUNICATIONS RESEARCH INSTITUTE, Daejeon (KR); Kyungpook National University Industry-Academic Cooperation Foundation, Daegu (KR)

(72) Inventors: Tae Wuk BAE, Daegu (KR); Yong Chul JUNG, Daegu (KR); Kyu Hyung KIM, Daegu (KR); Moon Kyu KIM, Daegu (KR); Jung Chul KIM, Daegu (KR); Jung Wook SUH, Daegu (KR); Eun Chang CHOI, Daegu (KR); Soo In LEE, Daegu (KR); Hyung Soo LEE, Daejeon (KR)

(21) Appl. No.: 15/867,488

(22) Filed: Jan. 10, 2018

(30) Foreign Application Priority Data
Jan. 10, 2017 (KR) 10-2017-0003467
Dec. 21, 2017 (KR) 10-2017-0177302

(51) Int. Cl. A61B 17/34

(52) U.S. Cl. CPC A61B 17/3468; A61B 17/3421

(57) ABSTRACT

An automatic hair follicle transplant apparatus of the present invention includes a housing, a needle supply portion, a needle transfer portion, a hair follicle insertion portion, a needle transfer operation portion, and an operation portion.

### CROSS-REFERENCE TO RELATED APPLICATION

This application claims priority to and the benefit of Korean Patent Application No. 10-2017-0003467, filed on Jan. 10, 2017, and 10-2017-0177302, filed on Dec. 21, 2017, the disclosure of which is incorporated herein by reference in its entirety.

### BACKGROUND

### 1. Field of the Invention

The present invention relates to an automatic hair follicle transplant apparatus, and more particularly, to an automatic hair follicle transplant apparatus capable of continuously supplying and withdrawing a needle in which a hair follicle is inserted using an external cam and link structure instead of an existing manual transplant apparatus.

### 2. Discussion of Related Art

In current hair transplantation, a nurse mounts only one hair follicle in each hair transplant needle and a surgeon who is a transplantation operator performs a hair transplant using a manual hair transplant apparatus in which the hair follicle is mounted.

In the case of a manual hair transplant apparatus currently used, since about 2,000 grafts are operated on one patient in one operation, fatigue of both the patient and a surgeon increases and 3 to 4 hours are consumed for the operation.

As described above, transplantation of hair follicles using a manual hair transplant apparatus has a lot of inconvenience in operation time or costs charged on the patient. Although, apparatuses capable of automatically/semiautomatically transplanting a hair follicle have been studied for overcoming the limitation, there is no case of succeeding in commercialization.

### SUMMARY OF THE INVENTION

Therefore, it is an aspect of the present invention to provide an automatic hair follicle transplant apparatus capable of continuously supplying a needle in which a hair follicle is inserted, using a needle supply portion, a needle transfer portion, a hair follicle insertion portion, a needle transfer operation portion, and an operation portion mounted in a housing.

It is another aspect of the present invention to provide an automatic hair follicle transplant apparatus capable of stably supplying and withdrawing a needle in which a hair follicle is inserted, by configuring a needle transfer operation portion in an external cam shape and mounting a needle withdrawing portion on a needle transfer portion.

According to one aspect of the present invention, an automatic hair follicle transplant apparatus includes a housing which includes an accommodation space formed therein, a needle through hole formed at a front, and a supply portion mounting hole formed in one side, a needle supply portion which is coupled with or separated from the housing through the supply portion mounting hole of the housing and supplies a needle with a hair follicle, a needle transfer portion which is mounted in the accommodation space of the housing and supplies or discharges the needle supplied by the needle supply portion by sliding, a hair follicle insertion portion which is disposed in the accommodation space of the housing and transplants the hair follicle accommodated in the needle into a scalp while moving forward, needle transfer operation portions which are arranged with an interval therebetween in a longitudinal direction in the accommodation space of the housing and slide the needle transfer portion through rotation of one ends, and an operation portion which is mounted in the accommodation space of the housing and operates the needle transfer portion, the hair follicle insertion portion, and the needle transfer operation portions in connection with operations of forward and backward driving motors.

A needle guide formed in a hollow shape with a conical end may be detachably mounted in the needle through hole.

The needle supply portion may include a needle supply case which is formed to vertically pass through an inside and with a needle mounting space formed therein, in which a plurality of needles are accommodated, a pressurizing spring mounting space which is formed to vertically pass through an inside of the needle supply case, a pressurizing spring mounted in the pressurizing spring mounting space, a pressurizing plate which is mounted in the needle mounting space located at a bottom of the pressurizing spring and pressurizes the needle, and a supply portion fixing device which is mounted on one side or both sides of the needle supply case and fixed to or separated from the housing.

A needle inflow guider may be mounted below the needle supply case. Here, the needle inflow guider may include a hinge, a pivoting member configured to pivot on the hinge and with a lower curved guide located below the needle supply case and a vertical pressurizing surface formed on top, a spring mounting groove fixed to an outer surface of the needle supply case, and a spring with one side supported by the spring mounting groove and the other side supported by the vertical pressurizing surface.

The pressurizing spring may include an upper elastic portion and a lower elastic portion integrally formed at a bottom of the upper elastic portion and formed to be narrower than the upper elastic portion.

The needle transfer portion may include fixing slides arranged on both sides on the basis of the needle through hole of the housing, operating slides which are arranged above the fixing slides, include needle support grooves formed in inner surfaces, and slide in connection with operations of the needle transfer operation portion and the operation portion, and buffering members mounted on parts at which the fixing slides and the operating slides are in contact with each other.

A needle withdrawing portion may be further mounted at a bottom of the needle transfer portion to withdraw and store the needle from which the hair follicle is removed. Here, the needle withdrawing portion may include upper links with tops coupled with lower slides located at the needle supply portion to operate in connection therewith, lower links with middle parts coupled with bottoms of the upper links, fixing shafts fixed to the housing and one ends of the lower links, a needle supporter which is mounted on the other ends of the lower links and supports the needle, and a withdrawing container which stores the withdrawn needle.

Front and rear support shafts may be mounted with an interval therebetween on the lower links. Here, an upper barrier bar may be mounted on the front support shaft to support the needle located at the needle supporter and also to prevent the needle from entering above. Also, a lower barrier bar may be mounted on the rear support shaft to prevent the needle located at the needle supporter from falling before a set angle.

The hair follicle insertion portion may include a hair follicle insertion portion cylinder, a coupling device mounted on an end of a rod of the hair follicle insertion portion cylinder, an operation portion connector with one end coupled with a bottom of the coupling device and the other end at which a bar coupling groove is formed, and a hair follicle pressurizing bar with one end coupled with the bar coupling groove of the operation portion connector and the other end which transplants the hair follicle accommodated in the needle into the scalp while moving forward.

The needle transfer operation portion may include two external cams, and the external cams are formed in different shapes.

The external cams may include a pair of rods formed in different shapes, rod fixing shafts fixed to one ends of the rods to the operation portion, and coupling rods with one ends coupled with the other ends of the rods and the other ends coupled with the slides of the needle transfer portion.

The operation portion may include a forward and backward driving motor on which a spiral shaft is mounted, an operation device which is mounted on the spiral shaft of the forward and backward driving motor and moves forward or backward according to forward and backward movement, an operation connecting bar which has one end coupled with the operation device and has a mounting space therein, in which the spiral shaft is accommodated, a rail connector formed at an end of the operation connecting bar, a needle transfer operation portion operation device which is coupled with the operation connecting bar and has both sides of a front, on which pressurizing rollers which move along inner surfaces of the needle transfer operation portions are mounted, a needle operation fixing device disposed in front of the operation connecting bar and in the needle transfer operation portion operation device and with a needle coupling member coupled with or separated from the needle and mounted on a front, a needle fixing operation device which is disposed in front of the needle operation fixing device and couples or separates the needle coupling member with or from the needle by moving the needle coupling member upward and downward when coming into contact with the needle coupling member, and a straight rail which is mounted in a longitudinal direction in the accommodation space of the housing, with a top with which the operation device is coupled, and which moves when the forward and backward driving motor operates.

The needle operation fixing device may include a pair of needle operation fixing device cases separately formed to be left and right, rotation operation needle fixing members mounted on top and bottom of the needle operation fixing device case to be symmetrical and with an outer surface formed in an atypical shape, and fixing member pressurizing springs which are mounted in the needle operation fixing device cases and pressurize the rotation operation needle fixing members.

The needle fixing operation device may include a lower support plate, a shape support portion formed in an L shape above the lower support plate, and needle fixing member operation sections formed on a top surface of the lower support plate and a bottom surface of the shape support portion to be collinearly symmetrical and formed of a curved surface, an incline, a horizontal surface, or a combination of a curve, an incline, and a horizontal surface.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a perspective view of an automatic hair follicle transplant apparatus according to one embodiment of the present invention;

FIG. 2 is a perspective view of the automatic hair follicle transplant apparatus according to one embodiment of the present invention from which an upper case is removed;

FIGS. 3 and 4 are a perspective view and a cross-sectional view illustrating a needle supply portion of the automatic hair follicle transplant apparatus according to one embodiment of the present invention;

FIGS. 5 to 7 are a perspective view, a separate perspective view, and a front view illustrating a needle transfer portion and a needle withdrawing portion of the automatic hair follicle transplant apparatus according to one embodiment of the present invention;

FIG. 8 is a view illustrating an operation state of the needle transfer portion and the needle withdrawing portion of the automatic hair follicle transplant apparatus according to one embodiment of the present invention;

FIG. 9 is a perspective view illustrating a hair follicle insertion portion of the automatic hair follicle transplant apparatus according to one embodiment of the present invention;

FIG. 10 is a perspective view illustrating a needle transfer operation portion and an operation portion of the automatic hair follicle transplant apparatus according to one embodiment of the present invention;

FIG. 11 is a perspective view illustrating the operation portion of the automatic hair follicle transplant apparatus according to one embodiment of the present invention;

FIGS. 12 and 13 are a perspective view and a rear perspective view illustrating a needle operation fixing portion and a needle fixing operation portion of the automatic hair follicle transplant apparatus according to one embodiment of the present invention;

FIGS. 14 and 15 are a plan view and a cross-sectional view illustrating a forward movement operation state of the automatic hair follicle transplant apparatus according to one embodiment of the present invention; and

FIGS. 16 and 17 are a plan view and a cross-sectional view illustrating a backward movement operation state of the automatic hair follicle transplant apparatus according to one embodiment of the present invention.

### DETAILED DESCRIPTION OF EXEMPLARY EMBODIMENTS

Advantages and features of the present invention and a method of achieving the same will become apparent with reference to the attached drawings and embodiments described below in detail. However, the present invention is not limited to the embodiments described below and may be embodied with a variety of different modifications. The embodiments are merely provided to allow one of ordinary skill in the art to completely understand the scope of the present invention and are defined by the claims. Meanwhile, the terms used herein are for explaining embodiments but not intended to limit the present invention. Throughout the specification, unless particularly defined otherwise, singular forms include plural forms. The terms “comprises” and/or “comprising” are used herein as meanings which do not exclude presence or addition of one or more other components, stages, operations, and/or elements in addition to stated components, stages, operations, and/or elements.

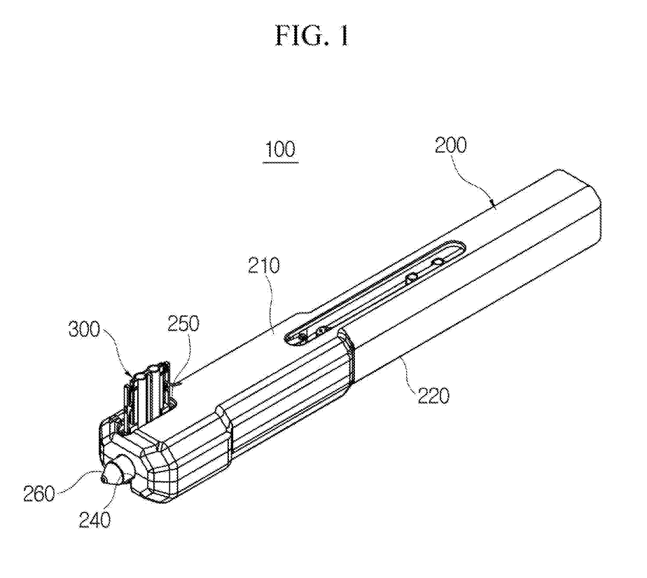

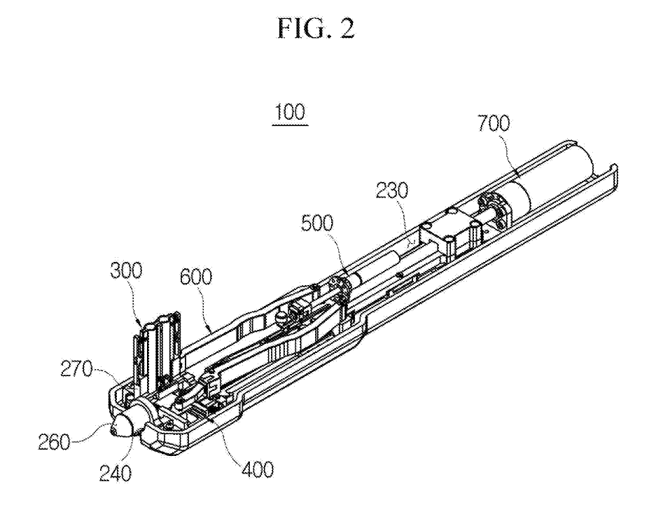

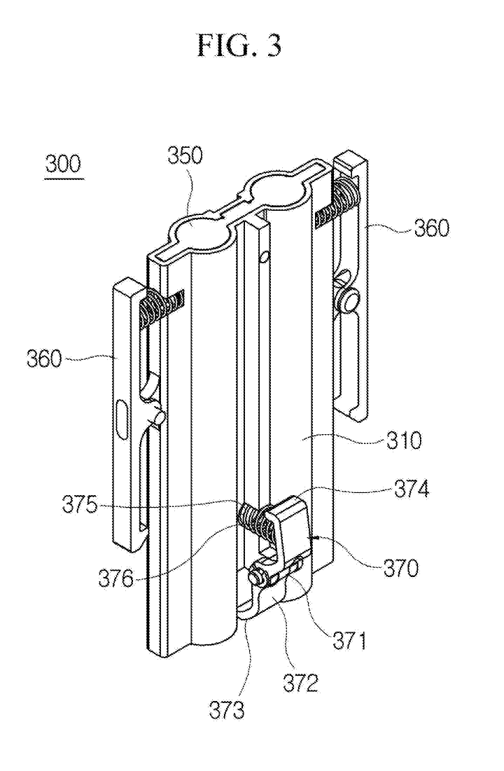

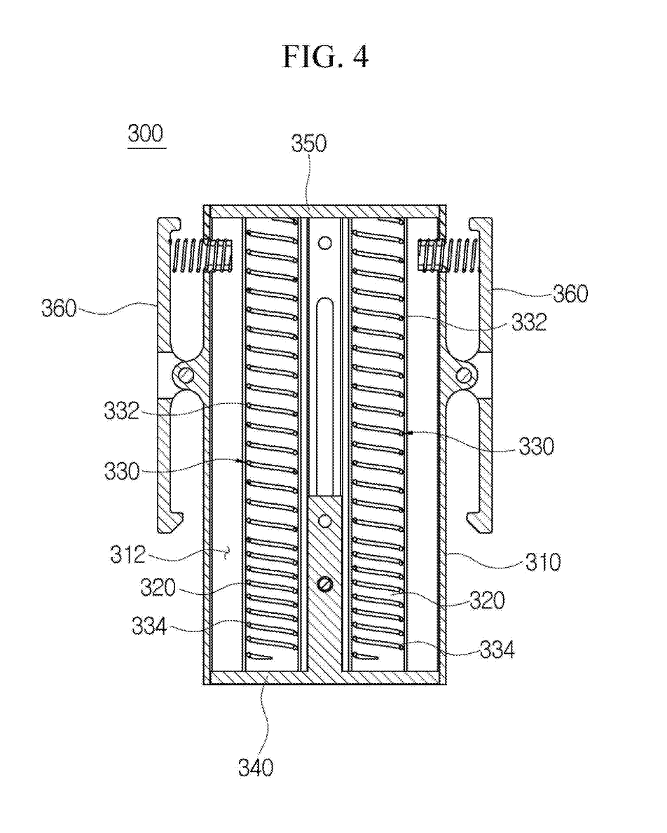

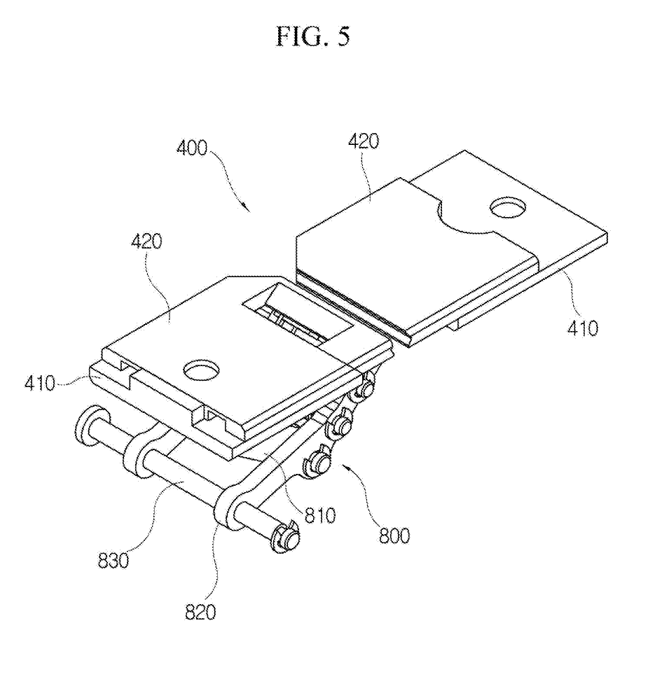

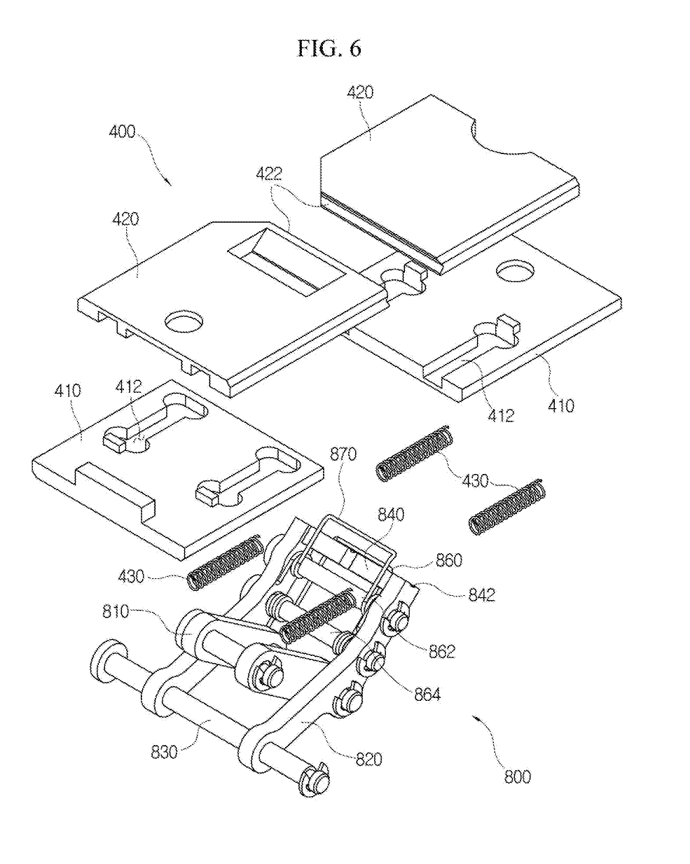

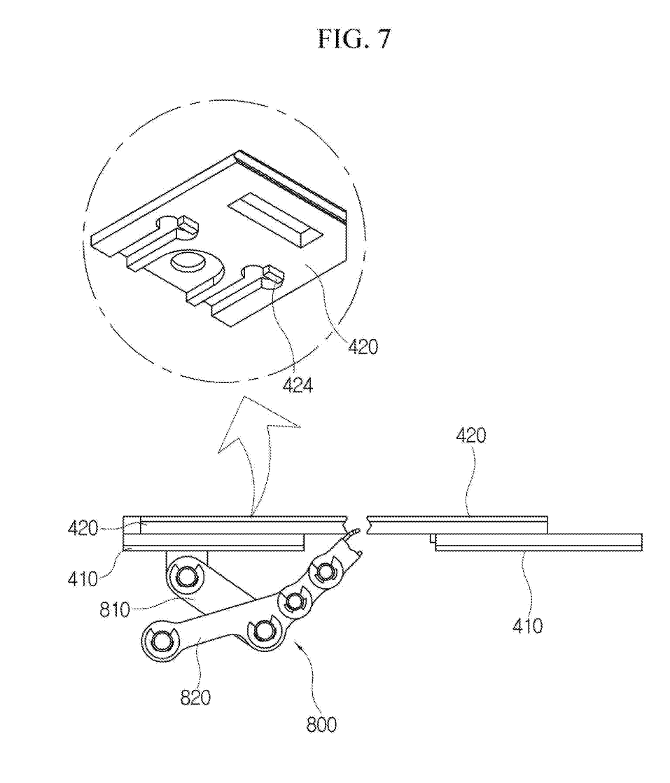

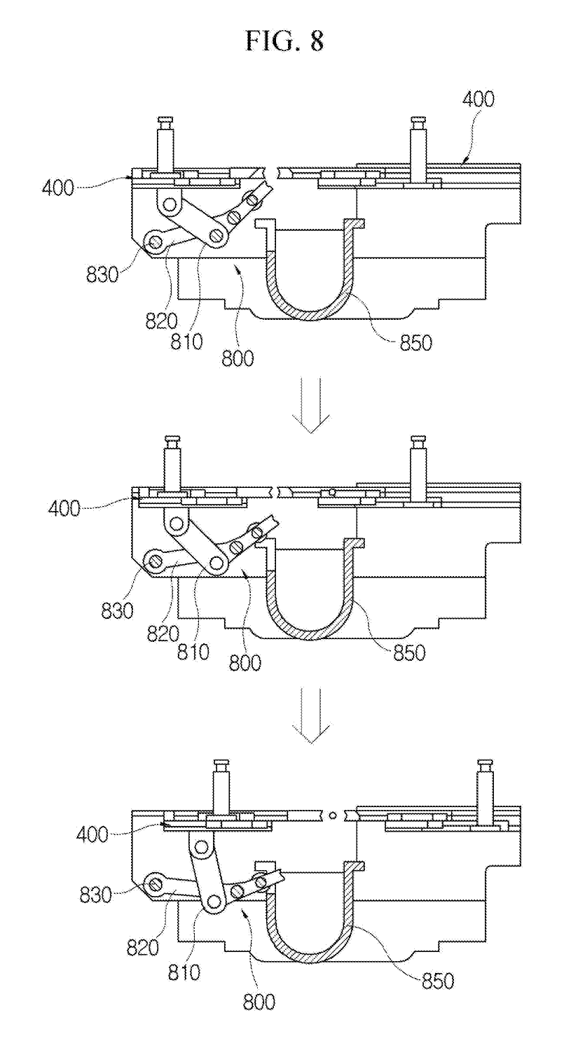

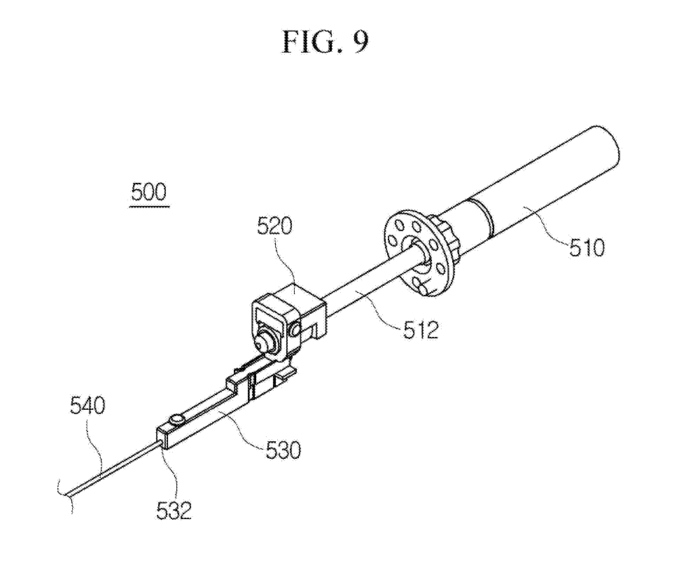

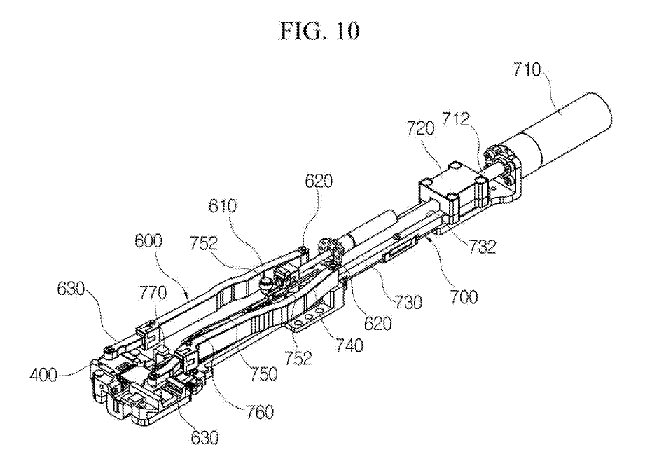

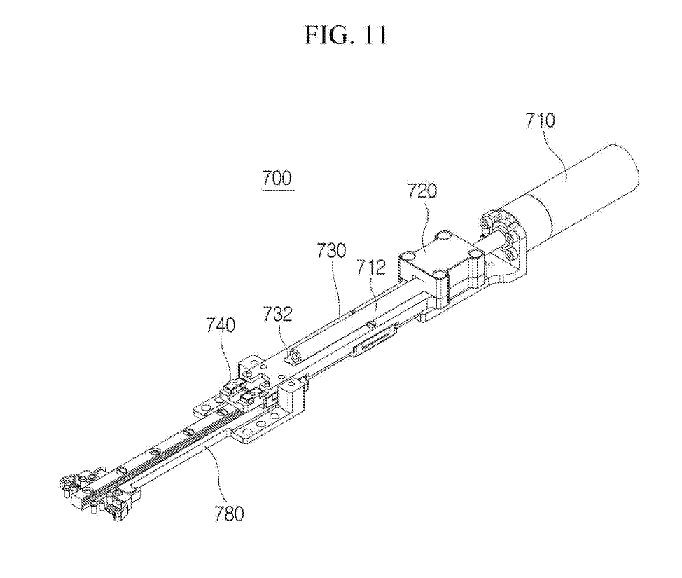

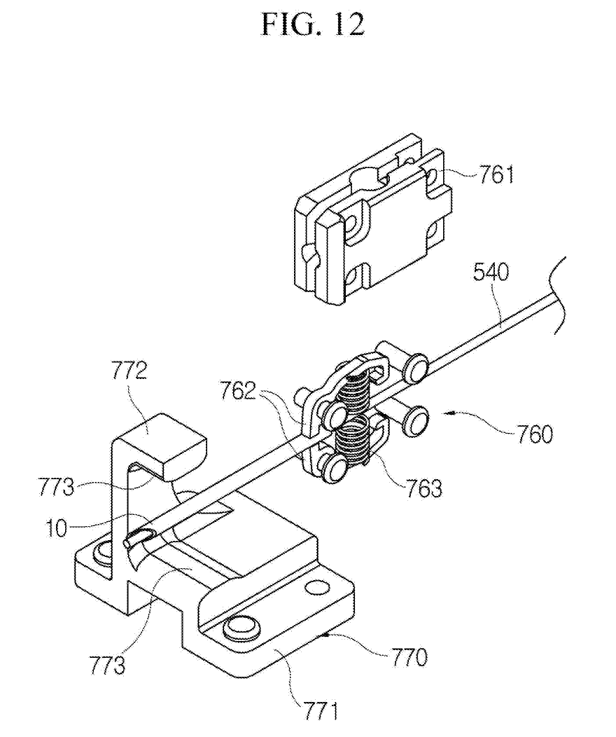

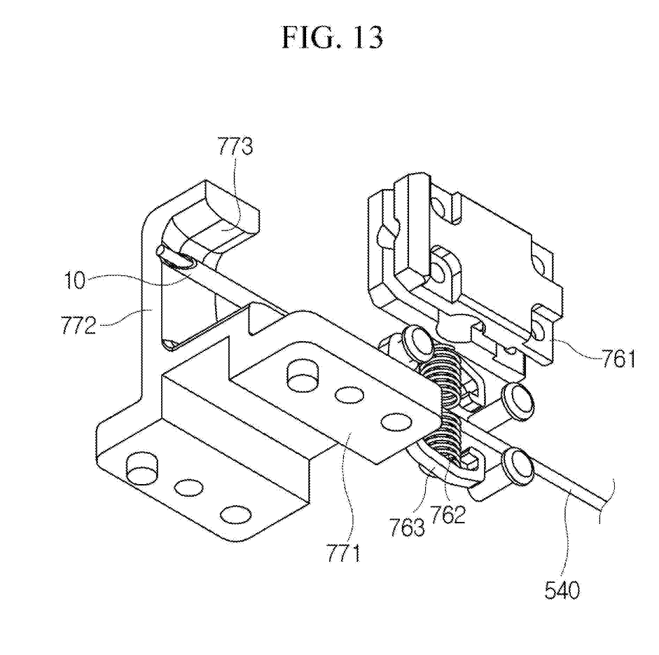

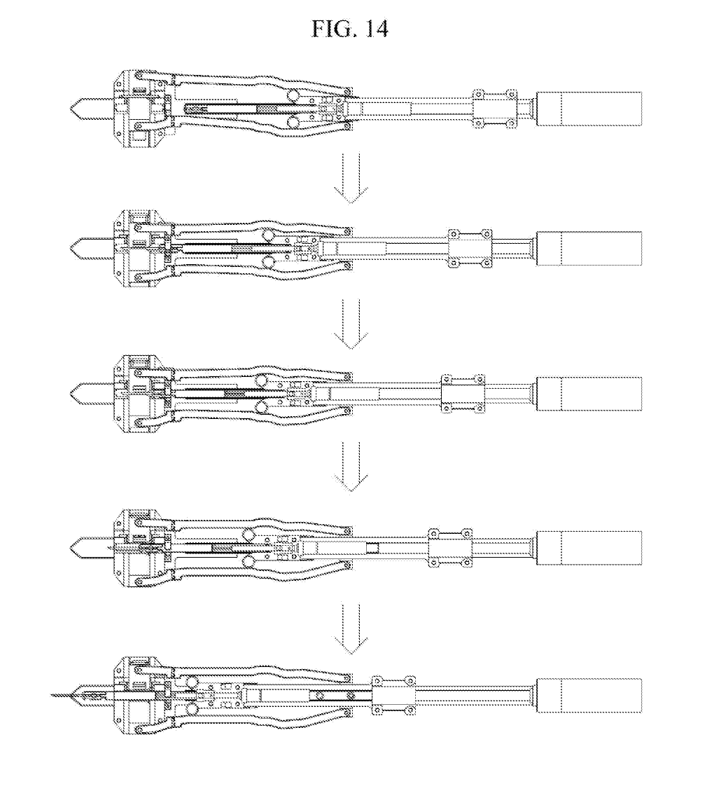

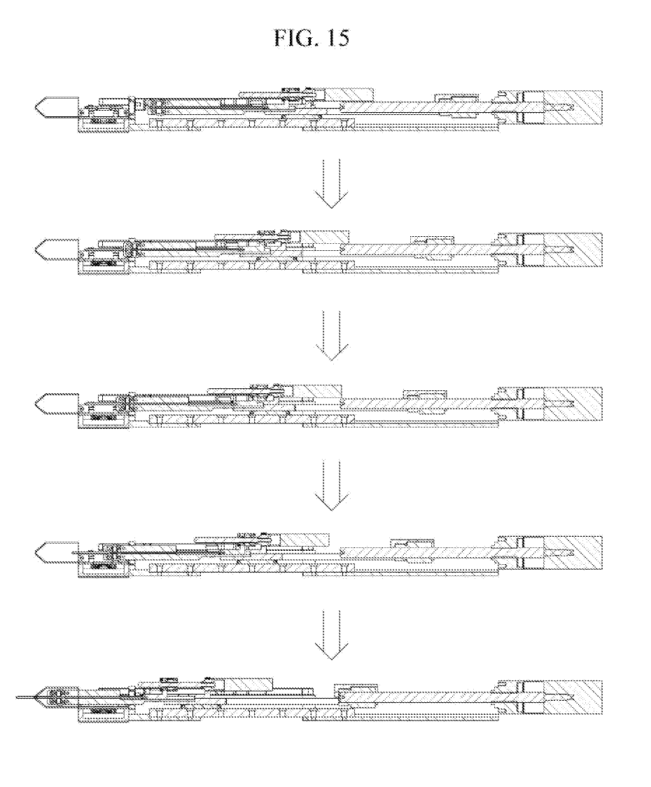

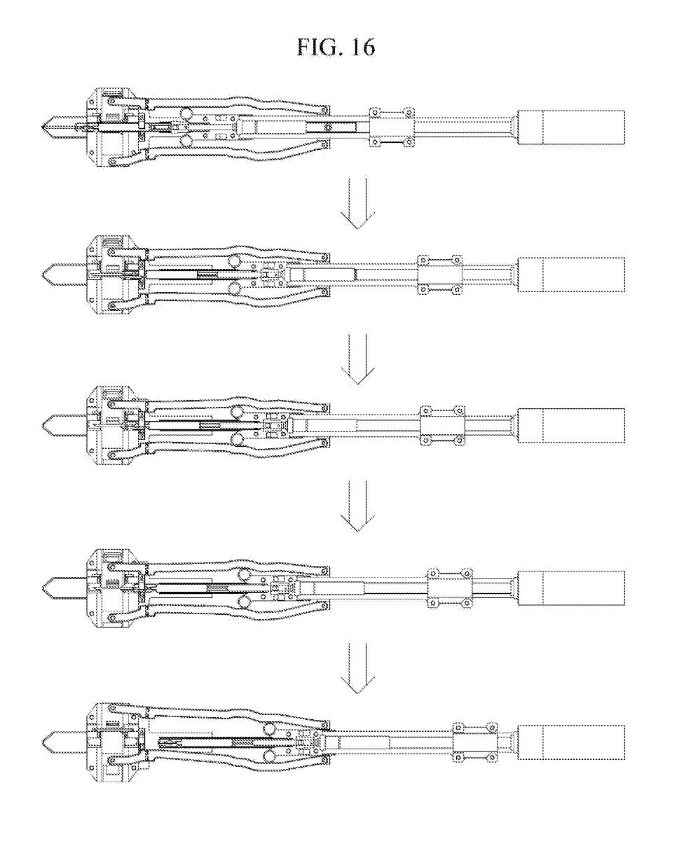

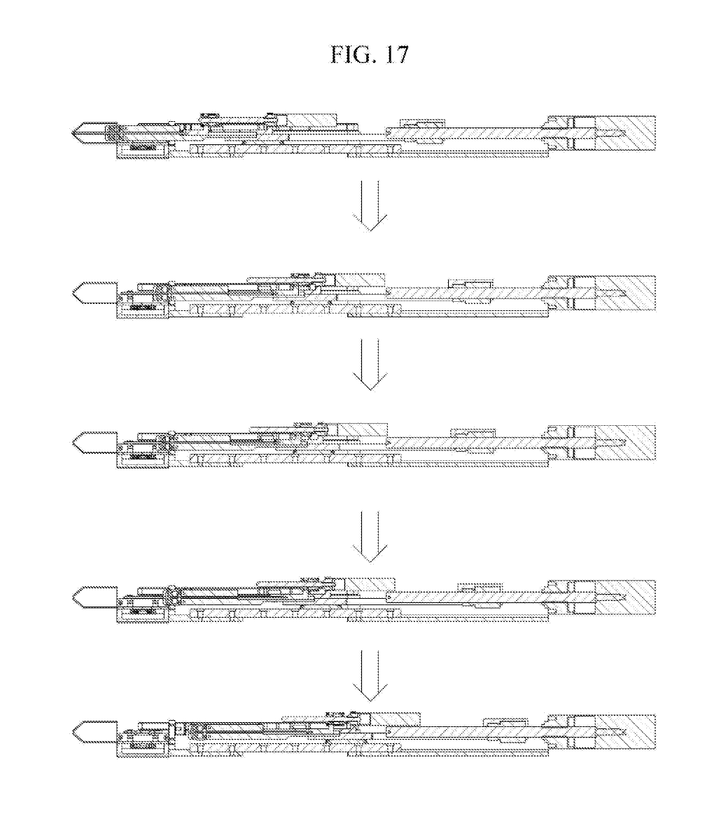

Hereinafter, exemplary embodiments of the present invention will be described in detail with reference to the attached drawings. FIG. 1 is a perspective view of an automatic hair follicle transplant apparatus according to one embodiment of the present invention. FIG. 2 is a perspective view of the automatic hair follicle transplant apparatus according to one embodiment of the present invention from which an upper case is removed. FIGS. 3 and 4 are a perspective view and a cross-sectional view illustrating a needle supply portion of the automatic hair follicle transplant apparatus according to one embodiment of the present invention. FIGS. 5 to 7 are a perspective view, a separate perspective view, and a front view illustrating a needle transfer portion and a needle withdrawing portion of the automatic hair follicle transplant apparatus according to one embodiment of the present invention. FIG. 8 is a view illustrating an operation state of the needle transfer portion and the needle withdrawing portion of the automatic hair follicle transplant apparatus according to one embodiment of the present invention. FIG. 9 is a perspective view illustrating a hair follicle insertion portion of the automatic hair follicle transplant apparatus according to one embodiment of the present invention. FIG. 10 is a perspective view illustrating a needle transfer operation portion and an operation portion of the automatic hair follicle transplant apparatus according to one embodiment of the present invention. FIG. 11 is a perspective view illustrating the operation portion of the automatic hair follicle transplant apparatus according to one embodiment of the present invention. FIGS. 12 and 13 are a perspective view and a rear perspective view illustrating a needle operation fixing portion and a needle fixing operation portion of the automatic hair follicle transplant apparatus according to one embodiment of the present invention.

An automatic hair follicle transplant apparatus 100 according to one embodiment of the present invention includes a housing 200, a needle supply portion 300, a needle transfer portion 400, a hair follicle insertion portion 500, a needle transfer operation portion 600, and an operation portion 700.

The housing 200 has a certain size and has a variety of shapes in addition to a shape shown in the drawings.

Also, the housing 200 includes separate upper and lower cases 210 and 220 and has an accommodation space 230 therein, a needle through hole 240 is formed at a front thereof, and a supply portion mounting hole 250 is formed at any one of the upper and lower cases 210 and 220.

Here, it will be described as an example that the supply portion mounting hole 250 is formed at the upper case 210.

That is, the housing 200 separately includes the upper and lower cases 210 and 220 to be easily couplable and separable and has the accommodation space 230 therein to accommodate the needle transfer portion 400, the hair follicle insertion portion 500, the needle transfer operation portion 600, the operation portion 700, a needle withdrawing portion 800, and the like. A needle through hole 240 for forward and backward movement of a needle 10 in which a hair follicle is inserted is formed at a front of the housing 200, and a supply portion mounting hole 250 is formed at the upper case 210 to allow the needle 10 in which a hair follicle is inserted, to be attachable and detachable.

Also, a needle guide 260 which has a hollow shape with a conic end is detachably mounted in the needle through hole 240.

That is, the needle guide 260 is mounted in the needle through hole 240 to accommodate a component therein as well as to prevent foreign substances from flowing thereinto.

Also, a needle supply portion guide 270 is formed inside the lower case 220 at a position corresponding to the supply portion mounting hole 250 for detachable coupling with the needle supply portion 300.

The needle supply portion 300 is attached to or detached from the needle supply portion guide 270 through the supply portion mounting hole 250 of the housing 200.

That is, the needle supply portion 300 is mounted on the needle supply portion guide 270 of the housing 200 to continuously supply the needle 10 in which a hair follicle is provided.

The needle supply portion 300 includes a needle supply case 310 formed to vertically pass through an inside and to include a needle mounting space 312 in which a plurality of needles are accommodated, a pressurizing spring mounting space 320 formed to vertically pass through the inside of the needle supply case 310, a pressurizing spring 330 mounted in the pressurizing spring mounting space 320, a pressurizing plate 340 which is mounted in the needle mounting space 312 located below the pressurizing spring 330 and pressurizes the needle 10, a case cover 350 which shields an open top of the needle supply case 310, and a supply portion fixing device 360 mounted on one side or both sides of the needle supply case 310 and fixed to or separated from the housing 200.

That is, in the needle supply portion 300, the needle mounting space 312 and the pressurizing spring mounting space 320 are formed inside the needle supply case 310 formed to vertically pass through, the pressurizing plate 340 is inserted and mounted at a bottom of the needle mounting space 312, and the pressurizing spring 330 is mounted in the pressurizing spring mounting space 320 such that the needle 10 which accommodates a hair follicle and is inserted in the needle mounting space 312 is supplied to the needle transfer portion 400 using elasticity.

Here, one or more pressurizing spring mounting spaces 320 are formed in the needle supply case 310. In the embodiment, there are two pressurizing spring mounting spaces 320 formed at an interval therebetween.

Also, the needle supply portion 300 is detachably coupled with the needle supply portion guide 270 through the supply portion fixing device 360 which rotatably operates using a hinge and a spring.

Also, the pressurizing springs 330 may be formed at the same intervals as well as an upper elastic portion and a lower elastic portion may be formed with different intervals as illustrated in the drawings.

That is, when the pressurizing spring 330 is formed with a uniform width, initial power is strong. On the other hand, when the pressurizing spring 330 includes an upper elastic portion 332 and a lower elastic portion 334 formed with different intervals, the lower elastic portion 334 rarely operates and only the upper elastic portion 332 operates at an initial stage such that a great discharge force like that of the initial stage may be provided as well as the upper elastic portion 332 and the lower elastic portion 334 operate together when a final needle is discharged, to maintain elasticity of the initial stage by increasing elasticity which has decreased.

Also, a needle inflow guider 370 is mounted on a bottom of the needle supply case 310.

That is, the needle inflow guider 370 guides the needle 10 which accommodates a hair follicle to the needle transfer portion 400 as well as prevents the needle 10 from being discharged outward.

Also, the needle inflow guider 370 includes a hinge 371, a pivoting member 372 which pivots on the hinge 371 and includes a curved guide 373 formed at a bottom of the pivoting member 371 and located below the needle supply case 310 and a vertical pressurizing surface 374 formed at a top of the pivoting member 371, a spring mounting groove 375 fixed to an outer surface of the needle supply case 310, and a spring 376 which includes one side supported by the spring mounting groove 375 and the other side supported by the vertical pressurizing surface 374.

That is, the needle inflow guider 370 adjusts the curved guide 373 according to an operation of the pivoting member 372 which pivots on the hinge 371.

The needle transfer portion 400 is mounted in the accommodation space 230 of the housing 200.

That is, the needle transfer portion 400, repetitively, moves the needle 10, which accommodates a hair follicle and is supplied by the needle supply portion 300 mounted on the needle supply portion guide 270 of the housing 200, to the needle through hole 240 through slidable operation and discharges outward the needle 10 from which the hair follicle is removed.

Also, the needle transfer portion 400 includes a pair of lower slides 410 arranged on both sides on the basis of the needle through hole 240 of the housing 200, a pair of upper slides 420 which are arranged above the lower slides 410, include needle support grooves 422 formed at inner surfaces, and slide in connection with operations of the needle transfer operation portion 600 and the operation portion 700, and buffering members 430 mounted on parts at which the lower slides 410 and the upper slides 420 come into contact with each other.

That is, in the needle transfer portion 400, when the operation portion 700 operates, the upper slides 420 linearly move along the lower slides 410 such that the needle 10 which accommodates a hair follicle and is supplied by the needle supply portion 300 is moved to the needle through hole 240.

Here, the buffering members 430 perform a buffering function according to the operations of the lower and upper slides 410 and 420 as well as a moving function in connection with the lower and upper slides 410 and 420.

In more detail, the buffering members 430 are formed of springs and mounted on slide mounting grooves 412 of the lower slides 410 such that both ends thereof pressurize the slide mounting grooves 412 of the lower slides 410 and protrusions 424 of the upper slides 420.

Also, the needle withdrawing portion 800 is further mounted on a bottom of the needle transfer portion 400 to withdraw and store the needle 10 from which the hair follicle is removed.

That is, when the needle transfer portion 400 operates, the needle withdrawing portion 800 operates in connection with the needle transfer portion 400 and withdraws the needle 10 from which the hair follicle is removed.

Also, the needle withdrawing portion 800 includes upper links 810 with tops coupled with the lower slides 410 located at the needle supply portion 300 to operate in connection therewith, lower links 820 with middle portions coupled with bottom ends of the upper links 810, a fixing shaft 830 fixed to the housing 200 and one ends of the lower links 820, a needle supporter 840 mounted on the other ends of the lower links 820 and with a needle support groove 842 formed at an outer surface to support the needle 10, and a withdrawing container 850 which accommodates the withdrawn needle 10.

That is, in the needle withdrawing portion 800, when lower slides 410 operate on the basis of the fixing shaft 830 which fixes the lower links 820 and the housing 200, the upper links 810 and the lower links 820 sequentially operate in connection with each other in order to, repetitively, accommodate the needle 10 from which the hair follicle is removed, to discharge the needle 10 from which a hair follicle is removed, into the withdrawing container 850 in a next operation, and withdrawing the needle 10 from which another hair follicle is removed.

Here, front and rear support shafts 862 and 864 are mounted with an interval therebetween on the lower links 820, an upper barrier bar 870 is mounted above on the front support shaft 862 to support the needle 10 located at the needle supporter 840 as well as to prevent the needle 10 from entering above, and a lower barrier bar 880 is mounted on the rear support shaft 864 to prevent the needle 10 located at the needle supporter 840 from falling before than a set angle.

That is, the upper barrier bar 870 and the lower barrier bar 880 allow one needle 10 to be located at the needle supporter 840 as well as prevent the needle 10 located at the needle supporter 840 from falling before the set angle.

The hair follicle insertion portion 500 is mounted in the accommodation space 230 of the housing 200.

That is, the hair follicle insertion portion 500 is mounted in the accommodation space 230 located in a middle part of the housing 200 and transplants a hair follicle accommodated in the needle 10 into a scalp while moving forward.

Also, the hair follicle insertion portion 500 includes a hair follicle insertion portion cylinder 510, a coupling device 520 mounted on an end of a rod 512 of the hair follicle insertion portion cylinder 510, an operation portion connector 530 which includes one end coupled with a bottom of the coupling device 520 and the other end at which a bar coupling groove 532 is formed, and a hair follicle pressurizing bar 540 which includes one end coupled with the bar coupling groove 532 of the operation portion connector 530 and while moving forward the other end transplants a hair follicle accommodated in the needle 10 into a scalp.

That is, in the hair follicle insertion portion 500, the coupling device 520 is mounted on the rod 512 of the hair follicle insertion portion cylinder 510 and the operation portion connector 530 with which the hair follicle pressurizing bar 540 is coupled is mounted on the coupling device 520 such that a hair follicle is transplanted into a scalp when the hair follicle insertion portion 500 moves forward.

The needle transfer operation portions 600 are mounted with an interval therebetween in the accommodation space 230 of the housing 200.

That is, the needle transfer operation portions 600 are arranged with an interval therebetween in a longitudinal direction in the accommodation space 230 of the housing 200 and pivot on one ends to slidably operate the needle transfer portion 400 when the operation portion 700 operates.

Here, the needle transfer operation portions 600 include two external cams, and the external cams are formed in different shapes.

That is, the needle transfer operation portions 600 include external cams differently configured according to an environment and purpose in order to slidably operate the needle transfer portion 400 when the operation portion 700 operates.

Also, the external cams include a pair of rods 610 formed in different shapes, rod fixing shafts 620 which fix one ends of the rods 610 to the operation portion 700, and coupling rods 630 which include one ends coupled with the other ends of the rods 610 and the other end coupled with the upper slides 420 of the needle transfer portion 400.

That is, in the external cams, the coupling rods 630 are coupled with fronts of the rods 610 in different shapes, the one ends of the rods 610 are fixed to the operation portion 700, and the other ends of the coupling rods 630 are coupled with the upper slides 420 of the needle transfer portion 400.

Here, the rods 610 of the external cams may be formed in the same shape according to an environment, purpose, and the like.

The operation portion 700 is mounted in the accommodation space 230 of the housing 200.

Also, the operation portion 700, according to operations of forward and backward driving motors, operate the needle transfer portion 400, the hair follicle insertion portion 500, and the needle transfer operation portion 600 in connection with one another, in order to transplant a hair follicle accommodated in the needle 10 into a scalp.

Here, the operation portion 700 includes a forward and backward driving motor 710 on which a spiral shaft 712 is mounted, an operation device 720 which is mounted on the spiral shaft 712 of the forward and backward driving motor 710 and moves forward or backward according to forward and backward movement, an operation connecting bar 730 which has one end coupled with the operation device 720 and has a mounting space 732 therein, in which the spiral shaft 712 is accommodated, a rail connector 740 formed at an end of the operation connecting bar 730, a needle transfer operation portion operation device 750 which is coupled with the operation connecting bar 730 and the rail connector 740 and has both sides of a front, on which pressurizing rollers 752 which move along inner surfaces of the needle transfer operation portions 600 are mounted, a needle operation fixing device 760 disposed in front of the operation connecting bar 730 and in the needle transfer operation portion operation device 750 and with a rotation operation needle fixing members 762 coupled with or separated from the needle 10 and mounted on a front, a needle fixing operation device 770 which is disposed in front of the needle operation fixing device 760 and couples or separates the rotation operation needle fixing members 762 with or from the needle 10 by operating the needle operation fixing device 760 upward and downward when coming into contact with the needle operation fixing device 760, a straight rail 780 which is mounted in a longitudinal direction in the accommodation space 230 of the housing, with a top with which the operation device 720 is coupled, and which moves when the forward and backward driving motor 710 operates.

That is, in the operation portion 700, the operation device 720 moves forward or backward along the straight rail 780 when the forward and backward driving motor 710 operates, and simultaneously, the operation connecting bar 730, the needle transfer operation portion operation device 750, and the needle operation fixing device 760, which are directly or indirectly connected to the operation device 720, also operate in connection with the operation device 720.

In more detail, the operation connecting bar 730 moves forward or backward in the same direction as that in which the operation device 720 moves.

Also, the needle transfer operation portion operation device 750 moves forward or backward when the operation device 720 operates and the pressurizing rollers 752 located on both sides of the front pressurize the rods 610 of the needle transfer operation portions 600 to operate the needle transfer operation portions 600 to pivot upward or downward with respect to the rod fixing shafts 620 such that the lower and upper slides 410 and 420 of the needle transfer portion 400 are operated.

Also, the needle operation fixing device 760 moves forward or backward in the same direction when the operation device 720 operates, and moves upward or downward when coming into contact with the needle fixing operation device 770 to be fixed to or separated from the needle 10.

That is, the needle operation fixing device 760 fixes the needle 10 by moving upward and downward with respect to a part which comes into contact with the needle fixing operation device 770 while moving forward and is separated from the needle 10 by moving upward and downward with respect to a part which comes into contact with the needle fixing operation device 770 while moving backward.

Here, the needle operation fixing device 760 includes a pair of needle operation fixing device cases 761 separately formed to be left and right, rotation operation needle fixing members 762 mounted on top and bottom of the needle operation fixing device case 761 to be symmetrical and with an outer surface formed in an atypical shape, and fixing member pressurizing springs 763 which are mounted in the needle operation fixing device cases 761 and pressurize the rotation operation needle fixing members 762.

Also, the needle fixing operation device 770 includes a lower support plate 771, a shape support portion 772 formed in an L shape above the lower support plate 771, and needle fixing member operation sections 773 formed on a top surface of the lower support plate 771 and a bottom surface of the shape support portion 772 to be collinearly symmetrical and formed of a curved surface, an incline, a horizontal surface, or a combination of a curve, an incline, and a horizontal surface.

That is, the needle operation fixing device 760 moves forward or backward while the forward and backward driving motor 710 operates, and moves upward and downward to be fixed to or separated from the needle 10 when the rotation operation needle fixing members 762 come into contact with the needle fixing member operation sections 773.

An example of the automatic hair follicle transplant apparatus configured as described above will be described as follows.

First, there is formed the needle supply portion 300 which includes the needle supply case 310 formed to vertically pass through the inside and to include the needle mounting space 312 in which a plurality of needles are accommodated, the pressurizing spring mounting space 320 formed to vertically pass through the inside of the needle supply case 310, the pressurizing spring 330 mounted in the pressurizing spring mounting space 320, the pressurizing plate 340 which is mounted in the needle mounting space 312 located below the pressurizing spring 330 and pressurizes the needle 10, the case cover 350 which shields an open top of the needle supply case 310, the supply portion fixing device 360 mounted on one side or both sides of the needle supply case 310 and fixed to or separated from the housing, and the needle inflow guider 370 mounted on the bottom of the needle supply case 310.

Also, there is configured the housing 200 which includes the separate upper and lower cases 210 and 220, the accommodation space 230 therein, the needle through hole 240 formed at the front thereof, the supply portion mounting hole 250 formed at any one of the upper and lower cases 210 and 220, the supply portion mounting hole 250 formed at any one of the upper and lower cases 210 and 220, the needle guide 260 in a hollow shape with the conical end and detachably mounted on the needle through hole 240, and the needle supply portion guide 270 formed in the lower case 220 at a position corresponding to the supply portion mounting hole 250.

Also, the needle transfer portion 400 is mounted in the accommodation space 230 of the housing 200, at which the needle through hole 240 is formed. Here, the needle transfer portion 400 includes the pair of lower slides 410 arranged on both sides on the basis of the needle through hole 240 of the housing 200, the pair of upper slides 420 which are arranged above the lower slides 410, include needle support grooves 422 formed at inner surfaces, and slide in connection with operations of the needle transfer operation portion 600 and the operation portion 700, and the buffering members 430 mounted on parts at which the lower slides 410 and the upper slides 420 come into contact with one each other.

Here, the needle withdrawing portion 800 is mounted below the needle transfer portion 400. Here, the needle withdrawing portion 800 includes the upper links 810 with tops coupled with the lower slides 410 located at the needle supply portion 300, the lower links 820 with middle portions coupled with bottom ends of the upper links 810, the fixing shaft 830 fixed to one ends of the lower links 820 and the housing 200, the needle supporter 840 mounted on the other ends of the lower links 820 and with the needle support groove 842 formed at an outer surface to support the needle 10, the withdrawing container 850 which accommodates the withdrawn needle 10, the front and rear support shafts 862 and 864 mounted with an interval therebetween on the lower links 820, the upper barrier bar 870 mounted above on the front support shaft 862 to support the needle 10 located at the needle supporter 840 as well as to prevent the needle 10 from entering, and the lower barrier bar 880 mounted on the rear support shaft 864 to prevent the needle 10 located at the needle supporter 840 from falling before a set angle.

Next, the operation portion 700 is mounted in the accommodation space 230 of the housing 200, opposite a side at which the needle through hole 240 is formed. Here, the operation portion 700 includes the forward and backward driving motor 710 on which the spiral shaft 712 is mounted, the operation device 720 which is mounted on the spiral shaft 712 of the forward and backward driving motor 710 and moves forward or backward according to forward and backward operation, the operation connecting bar 730 which has one end coupled with the operation device 720 and has the mounting space 732 therein, in which the spiral shaft 712 is accommodated, the rail connector 740 formed at the end of the operation connecting bar 730, the needle transfer operation portion operation device 750 which is coupled with the operation connecting bar 730 and the rail connector 740 and has both sides of the front, on which the pressurizing rollers 752 which move along the inner surfaces of the needle transfer operation portions 600 are mounted, the needle operation fixing device 760 disposed in front of the operation connecting bar 730 and in the needle transfer operation portion operation device 750 and with the rotation operation needle fixing members 762 coupled with or separated from the needle 10 and mounted on the front, the needle fixing operation device 770 which is disposed in front of the needle operation fixing device 760 and couples or separates the rotation operation needle fixing members 762 with or from the needle 10 by operating the needle operation fixing device 760 upward and downward when coming into contact with the needle operation fixing device 760, the straight rail 780 which is mounted in a longitudinal direction in the accommodation space 230 of the housing, with the top with which the operation device 720 is coupled, and which moves when the forward and backward driving motor 710 operates.

Also, the hair follicle insertion portion 500 is mounted on the operation connecting bar 730 of the operation portion 700. Here, hair follicle insertion portion 500 includes the hair follicle insertion portion cylinder 510, the coupling device 520 mounted on the end of the rod 512 of the hair follicle insertion portion cylinder 510, the operation portion connector 530 which includes one end coupled with the bottom of the coupling device 520 and the other end at which the bar coupling groove 532 is formed, and the hair follicle pressurizing bar 540 which includes one end coupled with the bar coupling groove 532 of the operation portion connector 530 and the other end transplants a hair follicle accommodated in the needle 10 into a scalp.

Next, when the needle transfer operation portion 600, formed of the pair of external cams which include the pair of rods 610 formed in different, the rod fixing shafts 620 fixing one ends of the rods 610 to the operation portion 700, and the coupling rods 630 with one ends coupled with other ends of the rods 610 and the coupling rods 630 with the other ends coupled with the upper slides 420 of the needle transfer portion 400, is mounted in the accommodation space 230 of the housing 200, the automatic hair follicle transplant apparatus 100 is completely assembled.

Here, an order of assembling the automatic hair follicle transplant apparatus 100 may be different from the above.

Next, an operation process of the automatic hair follicle transplant apparatus 100 will be described as follows.

First, the needle supply portion 300 in which a plurality of such needles 10 with a hair follicle accommodated therein are accommodated is mounted on the needle supply portion guide 270 of the housing 200.

Next, when the operation portion 700 is operated to move forward by using the forward and backward driving motor 710, the needle transfer portion 400, the hair follicle insertion portion 500, and the needle transfer operation portion 600 move forward in connection therewith.

Also, a position of the needle transfer operation portion 600 is shifted by the pressurizing rollers 752 of the needle transfer operation portion operation device 750 of the operation portion 700 which moves forward.

Here, the needle transfer portion 400 coupled with the needle transfer operation portion 600 operates in connection therewith such that the needle 10 with a hair follicle and supplied by the needle supply portion 300 is moved to a position of the needle through hole 240.

Afterwards, an end of the needle 10 with a hair follicle and positioned in front of the needle operation fixing device 760 is fixed and then the needle 10 is inserted into the scalp by the needle transfer operation portion operation device 750 which moves forward.

Next, while the needle 10 is inserted, the hair follicle insertion portion cylinder 510 of the hair follicle insertion portion 500 is operated such that the hair follicle pressurizing bar 540 transplants the hair follicle accommodated in the needle 10 into the scalp to the maximum.

Also, when the hair follicle is transplanted into the scalp, the hair follicle insertion portion 500 is moved backward, and then the operation portion 700 is operated backward using the forward and backward driving motor 710, the needle transfer portion 400, the hair follicle insertion portion 500, and the needle transfer operation portion 600 are moved backward in connection therewith.

Here, the needle transfer operation portion operation device 750 passes through the operation fixing device 760 and is separated from the needle 10.

Next, in the needle withdrawing portion 800 located below the needle transfer portion 400, when lower slides 410 operate on the basis of the fixing shaft 830 which fixes the lower links 820 and the housing 200, the upper links 810 and the lower links 820 sequentially operate in connection with each other in order to, repetitively, accommodate the needle 10 from which the hair follicle is removed, to discharge the needle 10 from which a hair follicle is removed, into the withdrawing container 850 in a next operation, and withdrawing the needle 10 from which another hair follicle is removed.

As described above, the automatic hair follicle transplant apparatus according to the embodiment of the present invention may continuously supply the needle with a hair follicle inserted therein through the needle supply portion, the needle transfer portion, the hair follicle insertion portion, the needle transfer operation portion, and the operation portion, which are mounted in the housing.

According to the embodiments of the present invention, an automatic hair transplant apparatus provides effects of continuously supplying a needle in which a hair follicle is inserted, using a needle supply portion, a needle transfer portion, a hair follicle insertion portion, a needle transfer operation portion, and an operation portion and also increasing operational precision by reducing a volume and a weight.

Also, an automatic hair follicle transplant apparatus capable of stably supplying and withdrawing a needle in which a hair follicle is inserted, by configuring a needle transfer operation portion in an external cam shape and mounting a needle withdrawing portion on a needle transfer portion and also reducing operation time and costs according thereto.

Although the technical concept of the present invention has been exemplarily described above, a variety of modifications may be made by one of ordinary skill in the art without departing from the essential features of the present invention.

Accordingly, the above-described embodiments of the present invention are not intended to limit but to explain the technical concept of the present invention, and the scope of the present invention is not limited thereto. It should be understood that the scope of the present invention is defined by the following claims and equivalents thereof are included in the scope of the present invention.

What is claimed is:

1. An automatic hair follicle transplant apparatus comprising:
a housing which comprises an accommodation space formed therein, a needle through hole formed at a front, and a supply portion mounting hole formed in one side;
a needle supply portion which is coupled with or separated from the housing through the supply portion mounting hole of the housing and supplies a needle with a hair follicle;
a needle transfer portion which is mounted in the accommodation space of the housing and supplies or discharges the needle supplied by the needle supply portion by sliding;
a hair follicle insertion portion which is disposed in the accommodation space of the housing and transplants the hair follicle accommodated in the needle into a scalp while moving forward;
needle transfer operation portions which are arranged with an interval therebetween in a longitudinal direction in the accommodation space of the housing and slide the needle transfer portion through rotation of one ends; and
an operation portion which is mounted in the accommodation space of the housing and operates the needle transfer portion, the hair follicle insertion portion, and the needle transfer operation portions in connection with operations of forward and backward driving motors.

2. The automatic hair follicle transplant apparatus of claim 1, wherein a needle guide formed in a hollow shape with a conical end is detachably mounted in needle blocking preventer

3. The automatic hair follicle transplant apparatus of claim 1, wherein the needle supply portion comprises:
a needle supply case which is formed to vertically pass through an inside and with a needle mounting space formed therein, in which a plurality of needles are accommodated;
a pressurizing spring mounting space which is formed to vertically pass through an inside of the needle supply case;
a pressurizing spring mounted in the pressurizing spring mounting space;
a pressurizing plate which is mounted in the needle mounting space located at a bottom of the pressurizing spring and pressurizes the needle; and
a supply portion fixing device which is mounted on one side or both sides of the needle supply case and fixed to or separated from the housing.

4. The automatic hair follicle transplant apparatus of claim 3, wherein a needle inflow guider is mounted below the needle supply case, and
wherein the needle inflow guider comprises:
a hinge;
a pivoting member configured to pivot on the hinge and with a lower curved guide located below the needle supply case and a vertical pressurizing surface formed on top;
a spring mounting groove fixed to an outer surface of the needle supply case; and
a spring with one side supported by the spring mounting groove and the other side supported by the vertical pressurizing surface.

5. The automatic hair follicle transplant apparatus of claim 3, wherein the pressurizing spring comprises an upper elastic portion and a lower elastic portion integrally formed at a bottom of the upper elastic portion and formed to be narrower than the upper elastic portion.

6. The automatic hair follicle transplant apparatus of claim 1, wherein the needle transfer portion comprises:
fixing slides arranged on both sides on the basis of the needle through hole of the housing;
operating slides which are arranged above the fixing slides, comprise needle support grooves formed in inner surfaces, and slide in connection with operations of the needle transfer operation portion and the operation portion; and
buffering members mounted on parts at which the fixing slides and the operating slides are in contact with each other.

7. The automatic hair follicle transplant apparatus of claim 6, wherein a needle withdrawing portion is further mounted at a bottom of the needle transfer portion to withdraw and store the needle from which the hair follicle is removed, and
wherein the needle withdrawing portion comprises:
upper links with tops coupled with lower slides located at the needle supply portion to operate in connection therewith;
lower links with middle parts coupled with bottoms of the upper links;
fixing shafts fixed to the housing and one ends of the lower links;
a needle supporter which is mounted on the other ends of the lower links and supports the needle; and
a withdrawing container which stores the withdrawn needle.

8. The automatic hair follicle transplant apparatus of claim 7, wherein front and rear support shafts are mounted with an interval therebetween on the lower links,
wherein an upper barrier bar is mounted on the front support shaft to support the needle located at the needle supporter and also to prevent the needle from entering above, and
wherein a lower barrier bar is mounted on the rear support shaft to prevent the needle located at the needle supporter from falling before a set angle.

9. The automatic hair follicle transplant apparatus of claim 1, wherein the hair follicle insertion portion comprises:
a hair follicle insertion portion cylinder;
a coupling device mounted on an end of a rod of the hair follicle insertion portion cylinder;
an operation portion connector with one end coupled with a bottom of the coupling device and the other end at which a bar coupling groove is formed; and
a hair follicle pressurizing bar with one end coupled with the bar coupling groove of the operation portion connector and the other end which transplants the hair follicle accommodated in the needle into the scalp while moving forward.

10. The automatic hair follicle transplant apparatus of claim 1, wherein the needle transfer operation portion comprises two external cams, and the external cams are formed in different shapes.

11. The automatic hair follicle transplant apparatus of claim 11, wherein the external cams comprise:
a pair of rods formed in different shapes;
rod fixing shafts fixed to one ends of the rods to the operation portion; and
coupling rods with one ends coupled with the other ends of the rods and the coupling rods with the other ends coupled with the slides of the needle transfer portion.

12. The automatic hair follicle transplant apparatus of claim 1, wherein the operation portion comprises:
a forward and backward driving motor on which a spiral shaft is mounted;
an operation device which is mounted on the spiral shaft of the forward and backward driving motor and moves forward or backward according to forward and backward movement;
an operation connecting bar which has one end coupled with the operation device and has a mounting space therein, in which the spiral shaft is accommodated;
a rail connector formed at an end of the operation connecting bar;
a needle transfer operation portion operation device which is coupled with the operation connecting bar and has both sides of a front, on which pressurizing rollers which move along inner surfaces of the needle transfer operation portions are mounted;
a needle operation fixing device disposed in front of the operation connecting bar and in the needle transfer operation portion operation device and with a rotation operation needle fixing members coupled with or separated from the needle and mounted on a front;
a needle fixing operation device which is disposed in front of the needle operation fixing device and couples or separates the needle coupling member with or from the needle by moving the needle coupling member upward and downward when coming into contact with the needle coupling member; and
a straight rail which is mounted in a longitudinal direction in the accommodation space of the housing, with a top with which the operation device is coupled, and which moves when the forward and backward driving motor operates.

13. The automatic hair follicle transplant apparatus of claim 12, wherein the needle operation fixing device comprises:
a pair of needle operation fixing device cases separately formed to be left and right;
rotation operation needle fixing members mounted on top and bottom of the needle operation fixing device case to be symmetrical and with an outer surface formed in an atypical shape; and
fixing member pressurizing springs which are mounted in the needle operation fixing device cases and pressurize the rotation operation needle fixing members.

14. The automatic hair follicle transplant apparatus of claim 12, wherein the needle fixing operation device comprises:
a lower support plate;
a shape support portion formed in an L shape above the lower support plate; and
needle fixing member operation sections formed on a top surface of the lower support plate and a bottom surface of the shape support portion to be collinearly symmetrical and formed of a curved surface, an incline, a horizontal surface, or a combination of a curve, an incline, and a horizontal surface.
